(19) United States
(12) Patent Application Publication
(10) Pub. No.: US 2014/0187884 A1
(43) Pub. Date: Jul. 3, 2014

(54) SYSTEMS AND METHODS FOR ENSEMBLE AVERAGING IN PULSE OXIMETRY

(71) Applicant: COVIDIEN LP, Mansfield, MA (US)

(72) Inventors: Paul S. Addison, Scotland (GB); James N. Watson, Scotland (GB)

(73) Assignee: Covidien LP, Mansfield, MA (US)

(21) Appl. No.: 13/730,415

(22) Filed: Dec. 28, 2012

(51) Int. Cl. A61B 5/00; A61B 5/0205; A61B 5/1495; A61B 5/1455

(52) U.S. Cl. CPC A61B 5/7225; A61B 5/14551; A61B 5/0205; A61B 5/1495
USPC 600/324

(57) ABSTRACT

Various methods and systems for ensemble averaging signals in a pulse oximeter are provided. An ensemble averaging method includes receiving an ensemble average signal corresponding to an ensemble average of electromagnetic radiation signals detected from a blood perfused tissue of a patient and receiving a pulse signal corresponding to a pulse detected by the pulse oximeter. The method also includes scaling a width of the ensemble average signal, a width of the pulse signal, or both to produce a scaled ensemble average signal and a scaled pulse signal having a substantially uniform width. The method further includes ensemble averaging the scaled ensemble average signal and the scaled pulse signal to produce an updated ensemble average signal having the substantially uniform width.

### BACKGROUND

The present disclosure relates generally to pulse oximetry and, more particularly, to ensemble averaging of pulses in a detected waveform from a pulse oximeter.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring physiological characteristics. Such devices provide doctors and other healthcare personnel with the information they need to provide healthcare for their patients. As a result, such monitoring devices have become an indispensable part of modern medicine. One technique for monitoring certain physiological characteristics of a patient is commonly referred to as pulse oximetry, and the devices built based upon pulse oximetry techniques are commonly referred to as pulse oximeters.

A pulse oximeter is typically used to measure various physiological characteristics, such as the blood oxygen saturation of hemoglobin in arterial blood and the pulse rate of the patient. Measurement of these characteristics has been accomplished by use of a non-invasive sensor that passes light through a portion of a patient's blood perfused tissue and photo-electrically senses the absorption and scattering of light in such tissue. The amount of light absorbed and scattered is then used to estimate the amount of blood constituent in the tissue using various algorithms known in the art. The “pulse” in pulse oximetry comes from the time varying amount of arterial blood in the tissue during a cardiac cycle. The signal processed from the sensed optical measurement is the plethysmographic waveform, which corresponds to the cyclic attenuation of optical energy through a portion of a patient's blood perfused tissue.

Ensemble averaging is a temporal averaging scheme that may be utilized to combine similar signals or similar portions of the same signal in order to improve the signal-to-noise ratio of the acquired data. In a pulse oximeter, ensemble averaging is used to calculate a weighted average of new samples and previous ensemble-averaged samples from one pulse-period earlier, and this weighted average may be utilized to determine a desired blood characteristic. For example, during a typical ensemble averaging operation, different weights may be assigned to different pulses, and a composite, averaged pulse waveform may be used to determine blood oxygen saturation.

A variety of techniques have been developed to attempt to improve the obtainable signal-to-noise ratio when ensemble averaging is utilized in pulse oximetry. For example, because the weights used for ensemble averaging have a significant effect on the ensemble averaging process, some implementations base the selected weights on the characteristics of the signals that are being ensemble averaged. In one implementation, when a new sample is suspected to have a high signal-to-noise ratio, the weight of the new sample may be increased, and when a new sample is suspected to be noisy, the weight of the sample may be decreased. Unfortunately, while these techniques may be advantageous in certain instances, in other instances, the weighted average obtained via ensemble averaging may still be prone to averaging errors due to physiological factors. For example, when the heart rate of a patient varies with time, the ensemble average waveform and the new sample may have different lengths, thus blurring the computed ensemble average waveform. Accordingly, there exists a need for ensemble averaging techniques that address these drawbacks.

### BRIEF DESCRIPTION OF THE DRAWINGS

Advantages of the disclosed techniques may become apparent upon reading the following detailed description and upon reference to the drawings in which:

FIG. 1 illustrates an embodiment of a patient monitoring system including a patient monitor and a sensor;

FIG. 2 is a block diagram illustrating an embodiment of a patient monitoring system including a sensor and a pulse oximeter;

FIG. 3 is a flow chart illustrating an embodiment of an ensemble averaging method that may be implemented to ensemble average pulses in a detected waveform from a pulse oximeter;

FIG. 4 is a flow chart illustrating an embodiment of an ensemble averaging method that may be implemented to generate and update an ensemble average waveform throughout a pulse oximetry data collection operation;

FIG. 5 is a plot illustrating examples of pulse waveforms that may be detected by a pulse oximeter and examples of ensemble average waveforms that may be generated through traditional ensemble averaging methods;

FIG. 6 is a plot illustrating an example ensemble average waveform that may be generated through a traditional ensemble averaging method;

FIG. 7 is a plot illustrating a series of ensemble average waveforms that may be generated throughout a pulse oximetry data collection operation via an embodiment of a presently disclosed ensemble averaging method;

FIG. 8 is a plot illustrating an ensemble averaging waveform generated after a pulse oximetry data collection operation commences and having a width corresponding to the latest pulse acquired in the collection operation in accordance with an embodiment of the presently disclosed technique;

FIG. 9 is a plot illustrating a series of ensemble average waveforms that may be generated throughout a pulse oximetry data collection operation via an embodiment of a presently disclosed ensemble averaging method; and

FIG. 10 is a plot illustrating an ensemble averaging waveform generated after a pulse oximetry data collection operation commences and having a rescaled width in accordance with an embodiment of the presently disclosed technique.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, the methods and systems provided herein are directed toward the ensemble averaging of pulses in a detected waveform from a pulse oximeter. Presently disclosed embodiments include one or more features capable of accommodating pulses having different lengths during the ensemble averaging process. As compared to traditional processes, the disclosed ensemble averaging techniques may reduce or prevent the likelihood of blurring of the ensemble average waveform due to the averaging of pulses having different lengths. For example, in some embodiments, a patient's heart rate may be time-varying, thus giving rise to pulses having different lengths, and features of the disclosed methods may accommodate this physiological variability during the ensemble averaging of the detected pulses. The foregoing feature may improve the quality of the generated ensemble average waveform, thus possibly improving the likelihood that the ensemble average waveform may be utilized to accurately determine a physiological parameter of interest, such as blood oxygen saturation, blood pressure, pulse rate, and so forth.

Embodiments of the provided systems and methods for ensemble averaging may include features capable of decoupling the morphological and temporal averaging of components of each pulse of the detected waveform from the pulse oximeter. For example, a variety of linear and non-linear scaling methods may be utilized to rescale the width of the ensemble average waveform, the most recent pulse, or both, such that when the most recent pulse and the ensemble average waveform are combined, time axis uniformity has been established. Rescaling methods may include, for example, stretching or squeezing of the time axis of the ensemble average waveform to the width of the most recent pulse such that the morphological characteristics of the ensemble average waveform are preserved. This may be achieved in certain embodiments, for example, by identifying fiducial points (e.g., peaks, troughs, and so forth) and stretching or squeezing portions of the waveform between the identified fiducial points. However, a variety of linear and non-linear scaling methods may be utilized alone or in combination to establish a uniform time axis before ensemble averaging. Further, it should be noted that the particular scaling methods implemented in a given system by one skilled in the art are subject to a variety of implementation-specific variations.

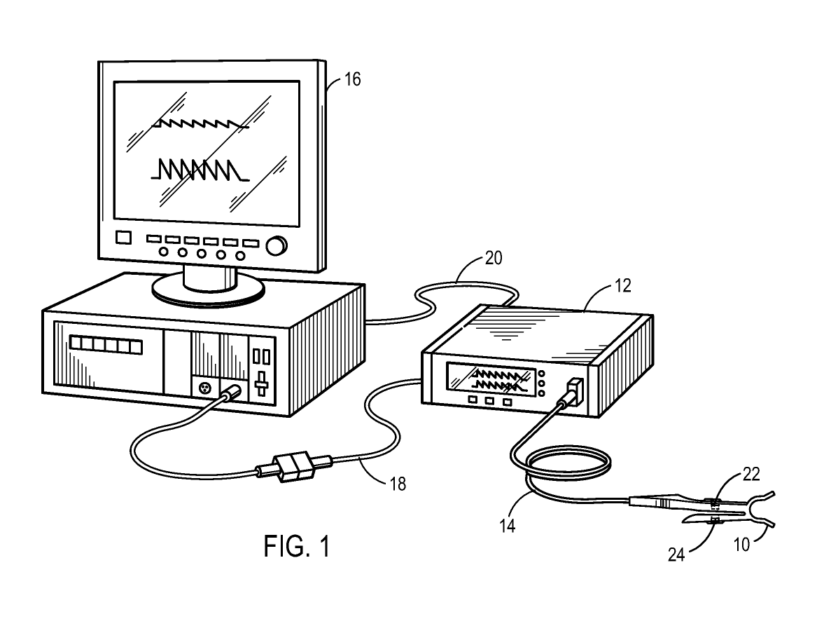

Turning now to the drawings, FIG. 1 illustrates a patient monitoring system that may utilize ensemble averaging in the process of monitoring a physiological characteristic of a patient. More specifically, the illustrated system may be capable of acquiring signals that correspond to detected waveforms from a sensor and further processing the signals to extract information that may be useful in the physiological monitoring process. To that end, the following description of the patient monitoring system serves as a basis for describing the ensemble averaging techniques described in more detail below.

The patient monitoring system of FIG. 1 includes a sensor 10 and a patient monitor 12. In the illustrated embodiment, a cable 14 connects the sensor 10 to the patient monitor 12. As will be appreciated by those of ordinary skill in the art, the sensor 10 and/or the cable 14 may include or incorporate one or more integrated circuit devices or electrical devices, such as a memory, processor chip, or resistor, that may facilitate or enhance communication between the sensor 10 and the patient monitor 12. Likewise, the cable 14 may be an adaptor cable, with or without an integrated circuit or electrical device, for facilitating communication between the sensor 10 and various types of monitors, including older or newer versions of the patient monitor 12 or other physiological monitors. In other embodiments, the sensor 10 and the patient monitor 12 may communicate via wireless means, such as using radio, infrared, or optical signals. In such embodiments, a transmission device (not shown) may be connected to the sensor 10 to facilitate wireless transmission between the sensor 10 and the patient monitor 12. As will be appreciated by those of ordinary skill in the art, the cable 14 (or corresponding wireless transmissions) are typically used to transmit control or timing signals from the monitor 12 to the sensor 10 and/or to transmit acquired data from the sensor 10 to the monitor 12. In some embodiments, however, the cable 14 may be an optical fiber that allows optical signals to be conducted between the monitor 12 and the sensor 10.

In one embodiment, the patient monitor 12 may be a suitable pulse oximeter, such as those available from Nellcor Puritan Bennett LLC. In other embodiments, the patient monitor 12 may be a monitor suitable for measuring tissue water fractions, or other body fluid related metrics, using spectrophotometric or other techniques. Furthermore, the monitor 12 may be a multi-purpose monitor suitable for performing pulse oximetry and measurement of tissue water fraction, or other combinations of physiological and/or biochemical monitoring processes, using data acquired via the sensor 10. Furthermore, to upgrade conventional monitoring functions provided by the monitor 12 to provide additional functions, the patient monitor 12 may be coupled to a multi-parameter patient monitor 16 via a cable 18 connected to a sensor input port and/or via a cable 20 connected to a digital communication port.

In embodiments in which the patient monitor 12 is a pulse oximeter, the pulse oximeter may be operated to detect a waveform having a variety of pulses. It may be desirable to utilize ensemble averaging to combine these pulses by averaging the most recent pulse with an ensemble average of the previous pulses throughout operation. Again, as described in more detail below, presently disclosed embodiments provide for establishment of uniformity of the length of the time axis of the most recent pulse and the ensemble average waveform before averaging, thus better preserving the morphological integrity of the pulses of the detected waveform during ensemble averaging. The foregoing feature offers an advantage over traditional systems in instances in which the lengths of the pulses in the detected waveform vary due to physiological factors (e.g., a patient's heart rate), thereby increasing the reliability of the ensemble average waveform when determining the physiological parameters of interest (e.g., blood oxygen saturation, heart rate, etc.).

In the example shown in FIG. 1, the sensor 10 is a clip-style sensor including an emitter 22 and a detector 24 which may be of any suitable type. For example, the emitter 22 may be one or more light emitting diodes capable of transmitting one or more wavelengths of light, such as in the red to infrared range, and the detector 24 may be a photodetector, such as a silicon photodiode package, selected to receive light in the range emitted from the emitter 22. In the illustrated embodiment, the sensor 10 is coupled to the cable 14 that is responsible for transmitting electrical and/or optical signals to and from the emitter 22 and detector 24 of the sensor 10. The cable 14 may be permanently or removably coupled to the sensor 10, depending on features of the implementation. For example, in instances in which the sensor 10 is disposable, the cable 14 may be removably coupled, for example, for cost efficiency purposes.

The sensor 10 described above is generally configured for use as a “transmission type” sensor for use in spectrophotometric applications, though in some embodiments it may instead be configured for use as a “reflectance type sensor.”Further, in other embodiments, the sensor 10 may be any suitable oximeter. For example, the sensor 10 may be an in-vivo optical spectroscopy oximeter capable of measuring changes in oxygen levels of a patient. Indeed, the sensor 10 may be any of a variety of types of sensors employed by those skilled in the art, not limited to the particular sensors that are described in detail herein.

Transmission type sensors include an emitter and detector that are typically placed on opposing sides of the sensor site. If the sensor site is a fingertip, for example, the sensor 10 is positioned over the patient's fingertip such that the emitter and detector lie on either side of the patient's nail bed. For example, the sensor 10 is positioned so that the emitter is located on the patient's fingernail and the detector is located opposite the emitter on the patient's finger pad. During operation, the emitter shines one or more wavelengths of light through the patient's fingertip, or other tissue, and the light received by the detector is processed to determine various physiological characteristics of the patient.

Reflectance type sensors generally operate under the same general principles as transmittance type sensors. However, reflectance type sensors include an emitter and detector that are typically placed on the same side of the sensor site. For example, a reflectance type sensor may be placed on a patient's fingertip such that the emitter and detector are positioned side-by-side. Reflectance type sensors detect light photons that are scattered back to the detector.

For pulse oximetry applications using either transmission or reflectance type sensors, the oxygen saturation of the patient's arterial blood may be determined using two or more wavelengths of light, most commonly red and near infrared wavelengths. Similarly, in other applications, a tissue water fraction (or other body fluid related metric) or a concentration of one or more biochemical components in an aqueous environment may be measured using two or more wavelengths of light, most commonly near infrared wavelengths between about 1,000 nm and about 2,500 nm. It should be understood that, as used herein, the term “light” may refer to one or more of infrared, visible, ultraviolet, or even X-ray electromagnetic radiation, and may also include any wavelength within the infrared, visible, ultraviolet, or X-ray spectra.

Pulse oximetry and other spectrophotometric sensors, whether transmission-type or reflectance-type, are typically placed on a patient in a location conducive to measurement of the desired physiological parameters. For example, pulse oximetry sensors are typically placed on a patient in a location that is normally perfused with arterial blood to facilitate measurement of the desired blood characteristics, such as arterial oxygen saturation measurement (SpO₂). Common pulse oximetry sensor sites include a patient's fingertips, toes, forehead, or earlobes. Regardless of the placement of the sensor 10, the reliability of the pulse oximetry measurement is related to the accurate detection of transmitted light that has passed through the perfused tissue and has not been inappropriately supplemented by outside light sources or modulated by subdermal anatomic structures. Such inappropriate supplementation and/or modulation of the light transmitted by the sensor can cause variability in the resulting pulse oximetry measurements.

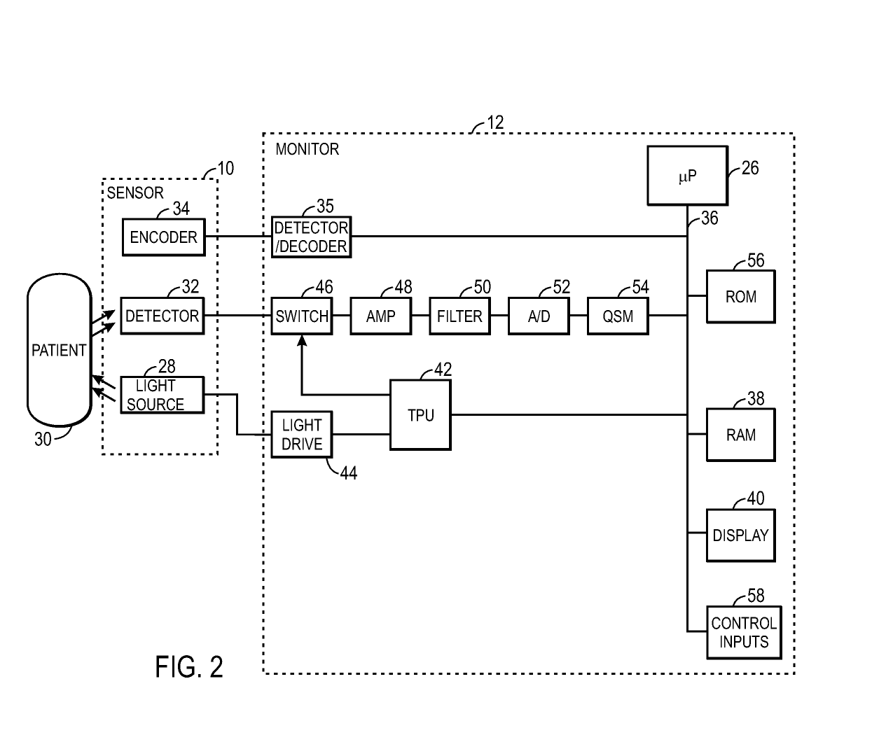

FIG. 2 is a block diagram of an embodiment in which the patient monitor is a pulse oximeter 12 that may be capable of implementing presently disclosed embodiments. That is, various embodiments of the presently disclosed ensemble averaging methods may be implemented as data processing algorithms that are executed by a microprocessor 26, which is provided as a component of the pulse oximeter 12 in the illustrated embodiment. Further, it should be noted that the embodiments of the present invention may be implemented as a part of a larger signal processing system used to process signals for the purpose of determining a desired physiological characteristic. As such, the microprocessor 26 may be operated alone or in conjunction with other processors in the signal processing system to implement the presently disclosed ensemble averaging methods. Again, presently contemplated algorithms that the microprocessor 26 may execute are described in more detail below.

Turning now to operation of the illustrated system, light from a light source 28 passes into a blood perfused tissue of a patient 30 and is scattered and detected by photodetector 32. The sensor 10 containing the light source 28 and the photodetector 32 may also contain an encoder 34 that provides signals indicative of the wavelength of light source 28 to a decoder 35 to allow the pulse oximeter 12 to select appropriate calibration coefficients for calculating oxygen saturation. In some embodiments, the encoder 34 may, for example, be a resistor. For further example, in other embodiments, the encoder 34 may be a memory device.

The sensor 10 is connected to the pulse oximeter 12. The pulse oximeter 12 includes the microprocessor 26 connected to an internal bus 36. A random access memory (RAM) memory 38 and a display 40 are also connected to the bus 36. A time processing unit (TPU) 42 provides timing control signals to light drive circuitry 44, which controls when light source 28 is illuminated and, if multiple light sources are used, the multiplexed timing for the different light sources. The TPU 42 also controls the gating-in of signals from photodetector 32 through a switching circuit 46. These signals are sampled at the proper time, depending upon which of multiple light sources is illuminated, if multiple light sources are used. The received signal is passed through an amplifier 48, a low pass filter 50, and an analog-to-digital converter 52. The digital data is then stored in a queued serial module (QSM) 54, for later downloading to RAM 38 as QSM 54 approaching its capacity. In one embodiment, there may be multiple parallel paths of separate amplifier, filter and A/D converters for multiple light wavelengths or spectra received.

Based on the value of the received signals corresponding to the light received by photodetector 32, microprocessor 26 will calculate the desired blood characteristics, such as blood oxygen saturation, using various algorithms. These algorithms may require coefficients, which may be empirically determined, corresponding to, for example, the wavelengths of light used. These and other parameters, constants, and so forth, may be stored in a read only memory (ROM) 56. In a two-wavelength system, the particular set of coefficients chosen for any pair of wavelength spectra is determined by the value indicated by encoder 34 corresponding to a particular light source in a particular sensor 10. Additionally, a variety of control inputs 58 may be utilized in the calculation of the desired blood characteristics. Control inputs 58 may be, for instance, a switch on the pulse oximeter, a keyboard, or a port providing instructions from a remote host computer. Furthermore, any number of methods or algorithms may be used to determine a patient's pulse rate, oxygen saturation or any other desired physiological parameter.

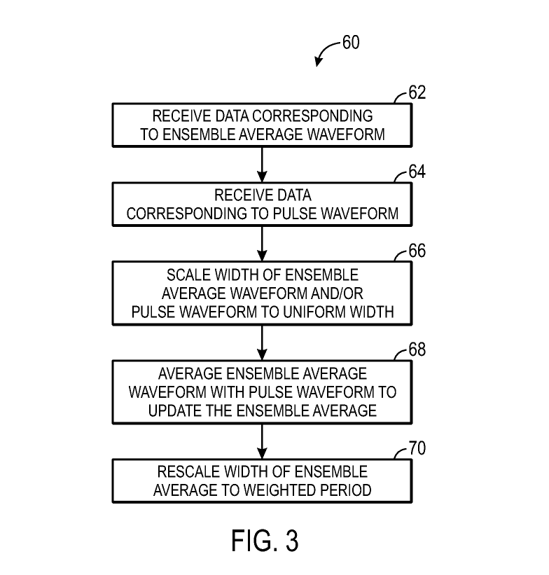

The brief description of the embodiment of the pulse oximeter 12 set forth above serves as a basis for describing presently disclosed embodiments of ensemble averaging methods for accommodating a width of the most recent pulse that is different from the width of the current ensemble average, which are described below in conjunction with FIG. 2. Specifically, FIG. 3 illustrates an embodiment of a method 60 that may be stored to memory and implemented by processing circuitry (e.g., microprocessor 26) to ensemble average a detected waveform from a pulse oximeter that has pulses of varying lengths, for example, due to a time-varying heart rate of a patient.

In particular, the method 60 includes receiving data corresponding to an ensemble average waveform (block 62). In some embodiments, the received ensemble average waveform may be an ensemble average of a variety of previously acquired pulses in the detected waveform acquired by the pulse oximeter. However, in other embodiments, the ensemble average waveform may be the waveform corresponding to a single pulse of the detected waveform, for example, during startup of the pulse oximeter when only a single pulse has been acquired at the time that the data is received. Still further, it should be noted that the ensemble average waveform may not be generated and displayed in some embodiments. Instead, in some embodiments, the values of the points in the ensemble average waveform may be stored and utilized for further processing.

The method 60 proceeds by receiving data corresponding to a pulse waveform (block 64). For example, the pulse waveform may correspond to a particular section of the waveform detected by the pulse oximeter 12 corresponding to the most recent pulse. That is, the pulse waveform may be derived from the detected waveform from the pulse oximeter 12. As previously mentioned, the period of the pulse waveform may differ from the period of the ensemble average waveform, for example, due to a time-varying heart rate. Accordingly, the method 60 proceeds by scaling the width of the ensemble average waveform and/or the width of the pulse waveform to a uniform width (block 66).

For example, in an instance in which the width of the pulse waveform is larger than the width of the ensemble average, a presently disclosed embodiment may provide for stretching of the time axis of the ensemble average waveform to the width of the pulse waveform. In such an embodiment, after the step of block 66 is performed, the uniform width of the ensemble average waveform and the pulse waveform is equal to the width of the received pulse waveform. For further example, in other embodiments, the time axis of the pulse waveform may be altered to match the width of the ensemble average waveform, or the time axes of both the pulse waveform and the ensemble average waveform may be squeezed or stretched to a uniform length not corresponding to the natural length of either waveform.

After a uniform time axis length has been established, the pulse waveform may be averaged with the ensemble average waveform to generate an updated ensemble average waveform (block 68). Again, because of the uniformity of the time axes of the pulse waveform and the ensemble average waveform, the averaging step of block 68 produces an updated ensemble average for which the likelihood of the introduction of noise due to mismatched periods is significantly reduced or eliminated. The foregoing feature may increase the reliability of the updated ensemble average as compared to traditional systems that may average waveforms having different widths, thus introducing noise and error to the updated ensemble average.

In some embodiments, the method 60 proceeds by rescaling the width of the updated ensemble average to a weighted period (block 70). This step may enable the morphological and temporal components of the pulse waveform to be independently ensemble averaged. For example, in one embodiment, the period of the updated and rescaled ensemble average (T_{D}) may be given by the following equation:

T_{D}=T_{D(old)}+wₜ*(Tₚ−T_{D(old)}) (1);

where T_{D(old)} is the previously determined uniform width, wₜ is the time rescaling weight, and Tₚ is the time period of the most recent pulse waveform. In this way, the weighted period to which the updated ensemble average is rescaled reflects a weighted average of the periods of the pulses that make up the ensemble average. It should be noted that the equation above is merely an example, and any of a variety of methods may be utilized by those skilled in the art to rescale the updated ensemble average to an appropriate weighted period. Further, the time rescaling weight may be determined based on a variety of physiological or operational factors, such as whether or not a sampling error occurred during that pulse, the presence of signal noise, the similarity of the pulse shape to previously received pulse shapes, and so forth.

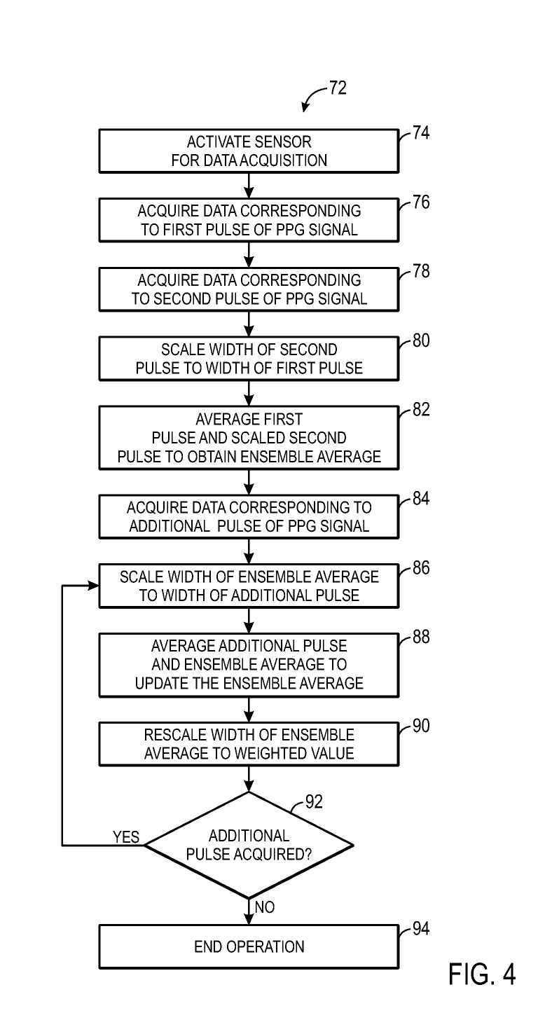

FIG. 4 illustrates an embodiment of a method 72 that may be implemented by a suitable controller, such as microprocessor 26, throughout a pulse oximetry monitoring operation to ensemble average a plurality of pulses in a detected waveform. The method 72 includes the step of activating the sensor (e.g., pulse oximeter 12) for data acquisition (block 74), for example, by turning on the sensor 10. The method 72 proceeds by acquiring data corresponding to a first pulse of the PPG signal (block 76) and a second pulse of the PPG signal (block 78). Once acquired, the width of the second pulse waveform is scaled to the width of the first pulse waveform (block 80), for example, by squeezing or stretching the time axis of the second pulse waveform to the time axis of the first pulse waveform. It should be noted that in other embodiments, the time axis of the first pulse waveform may be rescaled to the width of the second pulse waveform, or both the first pulse waveform and the second pulse waveform may be squeezed or stretched to a predetermined uniform width.

In the illustrated embodiment, after a uniform width has been established, the method 72 proceeds by averaging the first pulse waveform and the scaled second pulse waveform to generate an ensemble average waveform (block 82). As understood by those skilled in the art, the ensemble average waveform may be generated, for example, by assigning a weight to each pulse and combining the weighted pulses to generate the ensemble average. For example, in one embodiment, the new ensemble average pulse (P_{e(new)}) may be given by the following equation:

P_{e(new)}=[(1−wₐ)*Pₑ]+(wₐ*Pₚ) (2);

where wₐ is the assigned weight taking on a value between 0 and 1, Pₚ is the most recent pulse, and Pₑ is the current ensemble average. However, any of a variety of weighted or non-weighted ensemble averaging methods known to those skilled in the art may be employed to combine the first pulse waveform and the second pulse waveform in the step indicated by block 82.

The method 72 proceeds by acquiring data corresponding to an additional pulse of the PPG signal (block 84). That is, an additional pulse waveform is acquired, for example, by the pulse oximeter coupled to the patient. Here again, it should be noted that the additional pulse waveform may be an additional pulse of a single waveform, which may also include the first pulse waveform and the second pulse waveform, detected by the pulse oximeter throughout operation. The method 72 proceeds by scaling the width of the ensemble average waveform to the width of the additional pulse waveform (block 86). In certain embodiments, however, the width of the additional pulse waveform may be scaled to the width of the ensemble average waveform, or both waveforms may be scaled to a predetermined width. Regardless of the chosen width, the step indicated by block 86 results in an additional pulse waveform and an ensemble average waveform having a uniform width.

Once a uniform width has been established, the additional pulse waveform and the ensemble average waveform are averaged to produce an updated ensemble average waveform (block 88). As before, the width of the updated ensemble average waveform may then be rescaled to a weighted period (block 90), thus enabling the morphological and temporal components of the pulse waveform to be independently ensemble averaged. The method 72 proceeds by checking for additional acquired pulses (query block 92) and if no additional pulses are acquired (e.g., the sensor has been deactivated and data collection commenced), the operation is ended (block 94). However, if additional pulses are acquired, the ensemble average is updated throughout the operation to reflect the additional data. That is, for each additional acquired pulse waveform, the ensemble average waveform is rescaled to the width of the additional pulse (block 86), averaged with the additional pulse waveform (block 88), and rescaled to a weighted value (block 90).

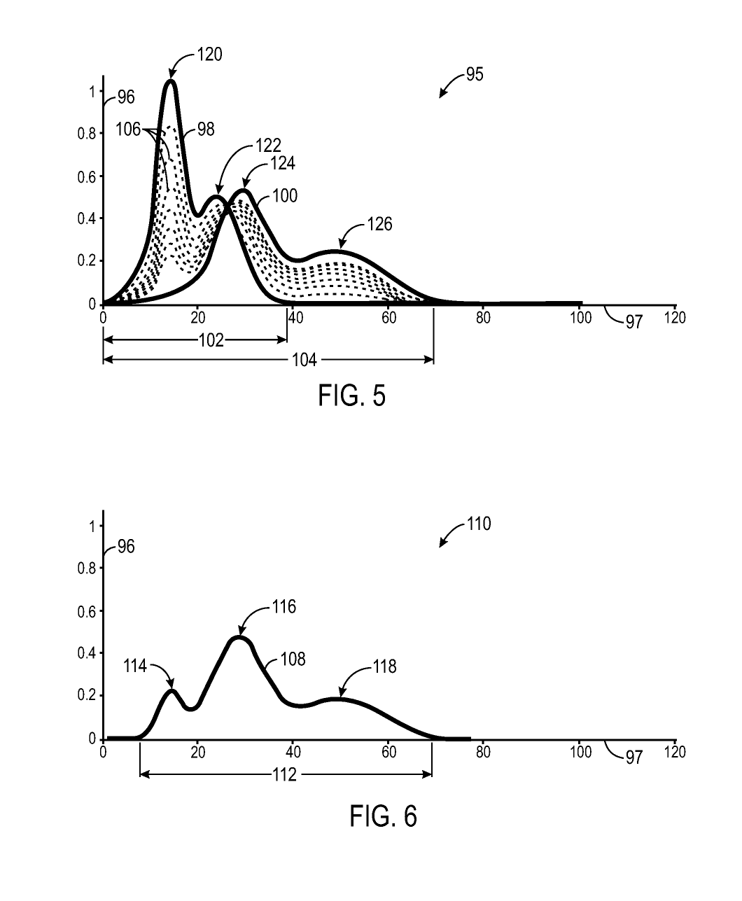

Embodiments of the foregoing methods 60 and 72 and the advantages of these methods over existing systems may be better understood through the following discussion of FIGS. 5-8. Specifically, FIG. 5 illustrates a plot 95 including a pulse waveform 98 and a pulse waveform 100 that may be acquired in an example pulse oximetry operation during which a pulse oximeter collects a series of pulse waveforms. The plot 95 includes an amplitude axis 96 and a time axis 97. In the example, the shapes of the acquired pulse waveforms transition over time from the pulse waveform 98 having a width 102 to the pulse waveform 100 having a width 104 throughout the collection of data by the pulse oximeter. In certain embodiments, the start of the waveforms shown in FIGS. 5, 7, 9 (e.g. waveform 98 or waveform 100) may be described by a trigger point determined from the waveform (e.g. a waveform's trough minimum). This trigger point may be used to synchronize the waveforms (e.g. waveform 98 or waveform 100) when computing an ensemble average, such as in the embodiment of the described method.

According to a traditional ensemble averaging method that does not accommodate for the changing width of the pulse waveforms during data collection, a series of intermediate ensemble average waveforms 106 may be generated throughout the pulse oximetry data collection operation. At the end of the pulse oximetry data collection operation, an ensemble average waveform 108 shown in plot 110 of FIG. 6 and having a width 112 is generated. The ensemble average waveform 108 represents the result of the ensemble averaging of the series of pulses acquired during the data collection operation by the pulse oximeter as the pulse shapes transitioned from the pulse waveform 98 to the pulse waveform 100. As shown in FIG. 6, the morphological characteristics of the acquired pulse waveforms 98 and 100 are not preserved in the ensemble average waveform 108. For example, the ensemble average waveform 108 includes three peaks 114, 116, and 118, while the pulse waveform 98 includes two peaks 120 and 122, and the pulse waveform 100 also includes two peaks 124 and 126.

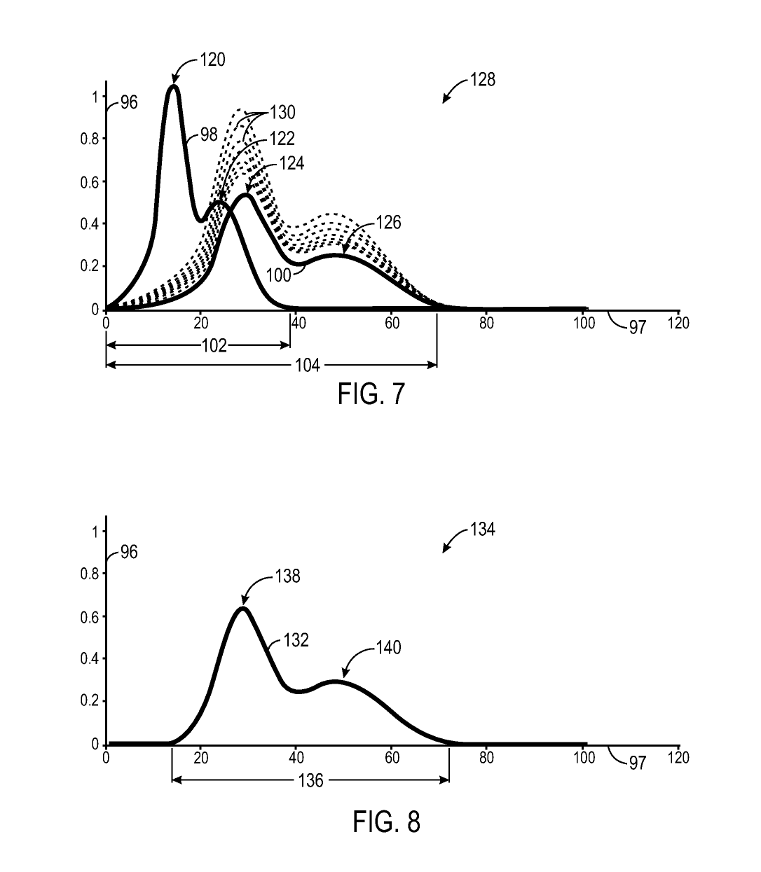

FIG. 7 illustrates a plot 128 that again includes the example pulse waveforms 98 and 100 acquired in a data collection operation in which the shapes of a series of pulse waveforms converge from the shape of waveform 98 to the shape of waveform 100 over time. However, by utilizing presently disclosed embodiments of ensemble averaging methods, such as the methods 60 and 72 described in FIGS. 3 and 4, a series of intermediate ensemble average waveforms 130 are generated throughout the pulse oximetry data collection operation. At the end of the pulse oximetry data collection operation, an ensemble average waveform 132 shown in plot 134 of FIG. 8 and having a width 136 is generated.

As shown in FIG. 8, by utilizing the presently disclosed ensemble averaging methods, the morphological characteristics of the pulse waveforms 98 and 100 are conserved throughout the ensemble averaging and are reflected in the ensemble average waveform 132. Specifically, as compared to the ensemble average waveform 108 obtained through traditional methods, the ensemble average waveform 132 obtained via presently disclosed embodiments includes only two peaks 138 and 140, thus better preserving the features of the pulse waveforms 98 and 100. Again, the morphological characteristics of the pulse waveforms 98 and 100 may be better preserved in presently disclosed embodiments because each time the ensemble average waveform is updated to include a newly acquired pulse, the widths of the ensemble average waveform and the new acquired pulse waveform are scaled to a uniform width.

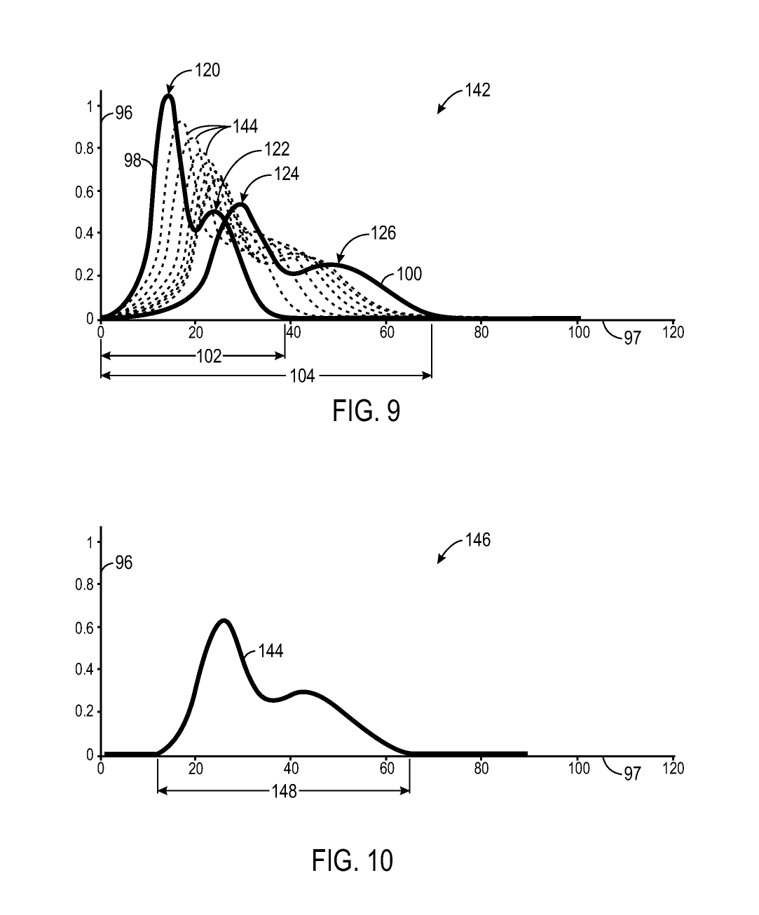

As in FIG. 7, a plot 142 shown in FIG. 9 includes the example pulse waveforms 98 and 100 acquired in a data collection operation in which the shapes of a series of pulse waveforms converge from the shape of the waveform 98 to the shape of the waveform 100 over time. However, in this embodiment, a series of intermediate ensemble average waveforms 144 are generated, and each of the waveforms in the series 144 is located in a different position along the time axis 97 with respect to the other waveforms in the series 144. That is, as compared to the series of intermediate ensemble average waveforms 130 of FIG. 7, the width of each of the ensemble average waveforms 144 shown in FIG. 9 has been rescaled to a weighted value after averaging with the most recent pulse waveform has been completed (e.g., as in the method step of block 70 of the method 60). Accordingly, at the end of the pulse oximetry data collection operation, an ensemble average waveform 144 shown in plot 146 of FIG. 10 is generated. A width 148 of the ensemble average waveform 144 of FIG. 10 is different than the width 136 of the ensemble average waveform 132 because the width 148 of the waveform 144 has been rescaled after the last pulse waveform has been averaged with the latest ensemble average waveform.

In some embodiments, the foregoing feature may enable the temporal component of the detected signal from the pulse oximeter to be decoupled from the morphological component of the detected signal, but for both the morphological and temporal components to be incorporated into the ensemble average waveform. Specifically, by rescaling the ensemble average waveform to the width of the latest pulse waveform before averaging, the morphological characteristics of the acquired pulse waveforms may be preserved in the ensemble average waveform. Additionally, the periods of the pulse waveforms may also be reflected in the ensemble average waveform, for example, by rescaling the ensemble average waveform to a weighted period that takes into account the periods of each of the pulse waveforms that have been averaged to form the ensemble average waveform.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A method of ensemble averaging signals in a pulse oximeter, comprising:
receiving an ensemble average signal corresponding to an ensemble average of electromagnetic radiation signals detected from a blood perfused tissue of a patient;
receiving a pulse signal corresponding to a pulse detected by the pulse oximeter;
scaling a width of the ensemble average signal, a width of the pulse signal, or both to produce a scaled ensemble average signal and a scaled pulse signal having a substantially uniform width; and
ensemble averaging the scaled ensemble average signal and the scaled pulse signal to produce an updated ensemble average signal having the substantially uniform width.

2. The method of claim 1, comprising scaling the updated ensemble average signal to a width corresponding to a weighted average of the periods of the electromagnetic radiation signals and the pulse signal.

3. The method of claim 1, wherein the scaling a width of the ensemble average signal, a width of the pulse signal, or both comprises scaling the width of the ensemble average signal, and wherein the substantially uniform width comprises the width of the pulse signal.

4. The method of claim 1, wherein the scaling a width of the ensemble average signal, a width of the pulse signal, or both comprises scaling the width of the pulse signal, and wherein the substantially uniform width comprises the width of the ensemble average signal.

5. The method of claim 1, wherein the ensemble averaging the scaled ensemble average signal and the scaled pulse signal comprises assigning a weight to the pulse signal, multiplying the weight by the pulse signal to produce a weighted pulse signal, and averaging the weighted pulse signal with the ensemble average signal.

6. The method of claim 1, wherein the pulse detected by the pulse oximeter corresponds to a time varying amount of arterial blood in the blood perfused tissue during a cardiac cycle of the patient.

7. A system, comprising:
a sensor comprising an emitter configured to transmit one or more wavelengths of light and a photodetector configured to receive the one or more wavelengths of light emitted by the emitter; and
a patient monitor configured to receive a detected signal from the sensor that corresponds to light received by the photodetector, wherein the patient monitor comprises:
processing circuitry configured to produce an ensemble average signal by ensemble averaging pulses of the detected signal, to receive a pulse signal from the sensor, and to produce an updated ensemble average signal by scaling a width of the ensemble average signal to a width of the received pulse signal and ensemble averaging the scaled ensemble average signal and the pulse signal.

8. The system of claim 7, wherein the patient monitor comprises a pulse oximeter.

9. The system of claim 7, wherein the processing circuitry is further configured to scale the updated ensemble average signal to a width corresponding to a weighted average of the periods of the pulses of the detected signal and the period of the received pulse signal.

10. The system of claim 7, wherein the patient monitor comprises memory configured to store the detected signal from the sensor, ensemble averaging code configured to be accessed by the processing circuitry, or both.

11. The system of claim 7, wherein the processor is further configured to calculate a blood characteristic based on the detected signal, the updated ensemble average signal, or both.

12. The system of claim 7, wherein the sensor comprises an encoder configured to provide signals to the patient monitor that correspond to the wavelength of the transmitted one or more wavelengths of light.

13. The system of claim 12, wherein the processing circuitry is further configured to utilize the provided signals to determine appropriate calibration coefficients for an oxygen saturation calculation.

14. A patient monitor system, comprising:
receiving circuitry configured to receive a detected signal corresponding to light received by a photodetector from a blood perfused tissue and to produce a processed signal; and
processing circuitry configured to receive the processed signal, to produce an ensemble average signal by ensemble averaging a first pulse signal and a second pulse signal included in the processed signal, and to produce an updated ensemble average signal by scaling a width of the ensemble average signal to a width of a third pulse signal included in the processed signal and ensemble averaging the scaled ensemble average signal and the third pulse signal.

15. The system of claim 14, wherein producing the processed signal comprises filtering the detected signal, amplifying the detected signal, performing an analog to digital conversion on the detected signal, or a combination thereof.

16. The system of claim 14, wherein the receiving circuitry comprises switching circuitry, amplification circuitry, filtering circuitry, an analog-to-digital converter, a queued serial module, or a combination thereof.

17. The system of claim 14, wherein the light received by the photodetector comprises electromagnetic radiation signals corresponding to at least two different wavelengths of light.

18. A tangible machine readable medium, comprising:
code configured to scale a width of an ensemble average signal, a width of a pulse signal, or both, to produce a scaled ensemble average signal and a scaled pulse signal having a substantially uniform width, wherein the ensemble average signal corresponds to an ensemble average of electromagnetic radiation signals detected from a blood perfused tissue of a patient and the pulse signal corresponds to a pulse detected by a pulse oximeter; and
code configured to ensemble average the scaled ensemble average signal and the scaled pulse signal to produce an updated ensemble average signal having the substantially uniform width.

19. The tangible machine readable medium of claim 18, comprising code configured to scale the updated ensemble average signal to a width corresponding to a weighted average of the periods of the electromagnetic radiation signals and the pulse signal.

20. The tangible machine readable medium of claim 18, wherein the code configured to ensemble average the scaled ensemble average signal and the scaled pulse signal is configured to assign a weight to the pulse signal, to multiply the weight by the pulse signal to produce a weighted pulse signal, and to average the weighted pulse signal with the ensemble average signal.
